(19) United States
(12) Patent Application Publication
(10) Pub. No.: US 2008/0275023 A1
(43) Pub. Date: Nov. 6, 2008

(54) N-Hydroxyamides Omege-Substituted with Tricyclic Groups as Histone Deacetylase Inhibitors, Their Preparation and Use in Pharmaceutical Formulations

(76) Inventors: Antonio Guidi, Firenze (IT); Tula Dimoulas, Empoli (IT); Danilo Giannotti, Altopascio (IT); Nicholas Harmat, Figline Valdarno (IT)

Correspondence Address: Jay S. Cinamon;Abelman Frayne & Schwab, 666 Third Avenue, 10th Floor, New York, NY, 10017-5621, US

(21) Appl. No.: 11/886,168

(22) PCT Filed: Mar. 13, 2006
(86) PCT No.: PCT/EP2006/060661

§ 371 Date: Sep. 10, 2007

(30) Foreign Application Priority Data
Mar. 15, 2005 (IT) FI2005A000042

(51) Int. Cl. A61K 31/554; C07D 243/14; C07D 285/36; A61P 3/10; A61K 31/7068; A61K 31/551

(52) U.S. Cl. 514/211.11; 540/557; 514/220; 540/547; 514/49

(57) ABSTRACT

New N-hydroxyamides of n-alkyl carboxylic acids omega substituted with suitable tricyclic systems characterised by a central 7-membered ring, having activity as inhibitors of histone deacetylase (HDAC).

### FIELD OF THE INVENTION

The present invention relates to omega substituted n-hydroxyamides of n-alkyl carboxylic acids which are inhibitory compounds of histone deacetylase, to preparations for obtaining them and to their use for the preparation of pharmaceutical formulations to be used in the treatment of pathologies in which the mechanism of gene regulation plays an essential role.

A particular aspect of the present invention is a compound having the general formula (I):

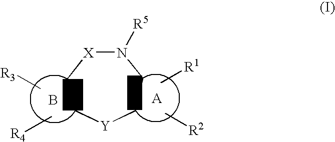

In which
X is chosen from the group: CO, CS, SO₂, CH₂
Y is chosen from the group: O, S, SO, SO₂, CH₂, C═O, C═CH₂, N—R6, CH—OR6, CH—NR6R9, C═CH—CO—R7

A and B are independently chosen from 5- or 6-membered rings, aromatics such as phenyl or heteroaromatics chosen from the group: furan, thiophene, pyrrole, oxazole, thiazole, imidazole, pyrazole, isoxazole, isothiazole, 1,2,3-oxathiazole, 1,2,3-triazole, pyridine, pyridazine, pyrimidine and pyrazine.
R1, R2, R3, R4 are independently chosen from the group: H, halogen, CF₃, NO₂, NR9R10, CN, COOH, (CH₂)m-CONR9R10, C1-6 alkyl, OH, O—C1-6 alkyl, O-cyclopropyl, O—(CH₂)2—O—C1-6 alkyl, O—(CH₂)2-NR9R10, O—CONHR9, CH₂-Z-R8, COR9, CR9R13R14, SR9, SOR15, SO₂R15, CR9NOR9, CR9NNR9R10, a Q-(CH₂)nCONHOH group, or a 5- or 6-membered ring chosen from the group: furan, thiophene, pyrrole, oxazole, thiazole, imidazole, pyrazole, isoxazole, isothiazole, 1,2,3-oxathiazole, 1,2,3-triazole, pyridine, pyridazine, pyrimidine, pyrazine, morpholine, thiomorpholine, piperidine, pyrrolidine
R5 and R6 can independently be a group chosen from: H, C1-6 alkyl, Q1-(CH₂)nCONHOH
R7 is a NH—(CH₂)nCONHOH group
R8 is a (CH₂)p-R11 group where R11 can be a methyl or a hydroxyl group
Z is chosen from the group O, NR12, S
Q can be a chemical bond, or can be chosen from the group —O—, —S—, —NR12-, —NR9CO—, —CONR9-, —W—, —COW— where W represents a group chosen from piperidine or pyrrolidine
Q1 can be a bond or a —CO—
R9 and R10 can independently be H or a C1-6 alkyl group
R12 is H or the R8 group
R13 and R14 can either both be a fluorine atom or oxygen atoms linked together by an alkyl chain consisting of 2 or 3 CH₂
R15 is a C1-6 alkyl
n is an integer between 2 and 9
m is an integer between 0 and 2
p is an integer between 0 and 5
   with the limitations that:
one group containing a (CH₂)nCONHOH hydroxamate and only one must always be present in the molecule
when X═CO and A and B both represent a benzene group, R3 and R4 cannot signify Q-(CH₂)nCONHOH.

All possible optical isomers, such as enantiomers and/or diastereoisomers, are also part of the present invention, derived from the possible presence of chiral centres or other stereogenic elements in compounds of general formula (I), and possible mixtures thereof, either as racemes or in various ratios thereof. Also equally included, when a group with basic or acid characteristic is present in the molecule, are salts with inorganic or organic acids or bases.

### STATE OF THE ART

Histone deacetylase is known to have an essential role in the mechanism that regulates gene expression. Inhibitors of histone deacetylase (HDAC) induce hyperacetylation of histones, with consequent alteration of gene expression itself. It follows that said inhibitors are useful as therapeutic or prophylactic agents for pathological states caused by abnormal gene expression, such as inflammatory disorders, diabetes, complications of diabetes, homozygotic thalassaemia, fibrosis, cirrhosis, acute promyelocytic leukaemia (APL), transplant rejection, auto-immune diseases, protozoan infections, tumors and the like.

The enzyme histone deacetylase is already well known and, via X-ray and SAR studies of various inhibitor classes, the structural characteristics that a potential inhibitor should possess have been elucidated; in particular a) a domain able to bind a metal (specifically Zn), b) a linker able to occupy a channel of the enzyme, c) a surface recognition domain that interacts with the structures on the rim of the enzyme active site (J. Med. Chem., 2003, 46(24), 5097-5116).

In the last few years many examples of HDAC inhibitors with the aforesaid structural characteristics have become apparent.

For example, compounds that present a N-hydroxyamide and a linear linker are described in: Bioorganic & Medicinal Chem Letters (2002), 12, 2919-2923; J Med Chem (2002) 45 (13), 2877-2885; J Med Chem (2002), 45 (4), 753-757; Bioorganic & Medicinal Chem Letters (2004), 14, 449-453. Other publications demonstrate hydroxamic acids in which the linker is not linear; in Bioorganic & Medicinal Chem Letters (2001), 11, 2847-2890 the linker is represented by a phenyl-ethyl or a styryl, in Bioorganic & Medicinal Chem Letters (2002), 12, 1347-1349 the linker is a phenyl or a cyclohexyl; the compounds described in WO2004013130 present a linker consisting of a thiophene.

Other authors have shown the possibility of substituting hydroxamic acid with other groups able to bind the metal of the enzyme active site, for example with amides (J. Med Chem (2003), 46, 820-830; or in EP847992) or electrophile ketones. WO2004069133 describes compounds in which, based on the aforementioned scheme, the metal binding group is represented by a phenylendiamine amide, and the linker by a heterocycle chosen from indole, benzothiophene or benzofuran.

WO02/085883 describes hydroxamate alkyls ω-substituted with tricyclic groups. Generally claimed therein are hydroxamates where the tricyclic group is represented by 6-5-6 or 6-7-6 systems in which the two 6-membered rings are always phenyl rings. Of the compounds prepared and described in the examples, only one compound with a 6-7-6 type tricyclic group is actually noted, presenting an oxepinone as the 7-membered central group; furthermore this compound has an inhibitory activity at 10 nm equal to 62% proving to be definitely the lowest of all the compounds assayed.

Notwithstanding all that is already known on the subject, there is still however a great need to identify new HDAC inhibitors that allow us to prepare new drugs for the treatment of the many pathologies that are potentially curable via this mechanism of action.

### DETAILED DESCRIPTION

The aim of the present invention is to provide new HDAC inhibitors of general formula (I), useful as drugs, and the pharmaceutical compositions that contain them as active ingredients for the treatment or prophylaxis of pathologies such as inflammatory disorders, diabetes, complications of diabetes, homozygotic thalassaemia, fibrosis, cirrhosis, acute promyelocytic leukaemia (APL), transplant rejection, auto-immune diseases, protozoan infections, tumors and the like.

A group of preferred compounds of the present invention are those of general formula (I) in which:
X is chosen from the group: CO, SO₂
Y is chosen from the group: O, S, SO, SO₂, CH₂, C═O, C═CH₂, N—R6, C═CH—CO—R7

A and B are independently chosen from 5- or 6-membered rings, aromatics such as phenyl or heteroaromatics chosen from the group: thiophene, pyrrole, oxazole, thiazole, imidazole, pyrazole, isoxazole, isothiazole, 1,2,3-oxathiazole, 1,2,3-triazole, pyridine.
R1, R2, R3, R4 are independently chosen from the group: H, halogen, CF₃, NO₂, NR9R10, CN, COOH, (CH₂)m-CONR9R10, C1-6 alkyl, OH, O—C1-6 alkyl, O-cyclopropyl, O—(CH₂)₂—O—C1-6 alkyl, O—(CH₂)₂—NR9R10, O—CONHR9, CH₂-Z-R8, COR9, CR9R13R14, SR9, SOR15, SO₂R15, CR9NOR9, CR9NNR9R10, a Q-(CH₂)nCONHOH group
R5 and R6 can independently be a group chosen from: H, CI-6 alkyl, Q1-(CH₂)nCONHOH
R7 is a NH—(CH₂)nCONHOH group
R8 is a (CH₂)p-R11 group where R11 can be a methyl or a hydroxyl group
Z is chosen from the group O, NR12, S
Q can be a chemical bond, or can be chosen from the group: —O—, —S—, —NR12-, —NR9CO—, —CONR9-, —COW—, where W represents a group chosen from piperidine or pyrrolidine
Q1 can be a bond or —CO-—
R9 and R10 can independently be H or a C1-6 alkyl group
R12 is H or the R8 group
R13 and R14 can either both be a fluorine atom or oxygen atoms linked together by an alkyl chain consisting of 2 or 3 CH₂
R15 is a C1-6 alkyl
n is an integer between 2 and 9
m is an integer between 0 and 2
p is an integer between 0 and 5

With the limitations that:
one group containing a (CH₂)nCONHOH hydroxamate and only one must always be present in the molecule
when X═CO and A and B both represent a benzene group, R3 and R4 cannot signify Q-(CH₂)nCONHOH.

Particularly preferred are compounds of general formula (I) in which:
X is chosen from the group: CO, SO₂
Y is chosen from the group: O, S, SO, SO₂, C═O, N—R6

A and B are independently chosen from 5- or 6-membered rings, aromatics such as phenyl or heteroaromatics chosen from the group: thiophene, pyrrole, oxazole, thiazole, imidazole, pyrazole, isoxazole, isothiazole, 1,2,3-oxathiazole, 1,2,3-triazole, pyridine
R1, R2, R3, R4 are independently chosen from the group: H, halogen, CF₃, NO₂, NR9R10, CN, C1-6 alkyl, OH, O—C1-6 alkyl, O—(CH₂)₂—NR9R10, CH₂-Z-R8, COR9, CR9R13R14, SR9, SOR15, SO₂R15, a Q-(CH₂)nCONHOH group
R5 and R6 can independently be a group chosen from: H, C1-6 alkyl, Q1-(CH₂)nCONHOH
R8 is a (CH₂)p-R11 group where R11 can be a methyl or a hydroxyl group
Z is chosen from the group O, NR12, S
Q can be a chemical bond, or can be chosen from the group: —O—, —S—, —NR12-, —NR9CO—, —CONR9-, —COW—, where W represents a group chosen from piperidine or pyrrolidine
Q1 can be a bond or —CO—
R9 and R10 can independently be H or a C1-6 alkyl group
R12 is H or the R8 group
R13 and R14 can either both be a fluorine atom or oxygen atoms linked together by an alkyl chain consisting of 2 or 3 CH₂
R15 is a C1-6 alkyl
n is an integer between 2 and 6
p is an integer between 0 and 5
   with the limitations that:
one group containing a (CH₂)nCONHOH hydroxamate and only one must always be present in the molecule
when X═CO and A and B both represent a benzene group, R3 and R4 cannot signify Q-(CH₂)nCONHOH.

In the present invention preferred meanings for C1-6 alkyl are groups chosen from: methyl, ethyl, propyl, isopropyl, n-butyl, 2-butyl, tert-butyl, pentyl, hexyl, 3-hexyl; halogen means a group chosen from F, Cl, Br, I.

The HDAC inhibitors of the present invention can be synthesised in accordance with reactions known in the state of the art (Hargrave K D et al. in J. Med Chem 1991, 34. 2231-2241; Giannotti D et al in J Med Chem 1991, 1356-1362; Press, J. B. J. Med. Chem., 1979, 22, 6, 725-731; CA 73:87951 (1970) JP-45015983), but can vary greatly on the basis of the series of synthesis steps needed to prepare the individual compounds summarized in general formula (I).

A descriptive scheme is given hereinafter by way of example.

In the case of the present invention it is critical that formation of the tricyclic system can be conducted, by way of example, following one of the paths described in schemes 1 and 2, or variations thereof known to the expert of the art.

### General Scheme:

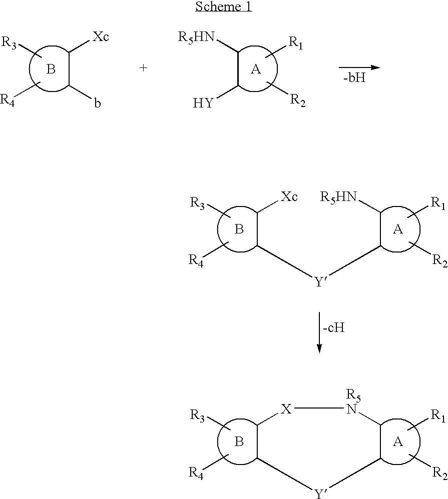

That described in general scheme 1 can be more easily followed in scheme 2

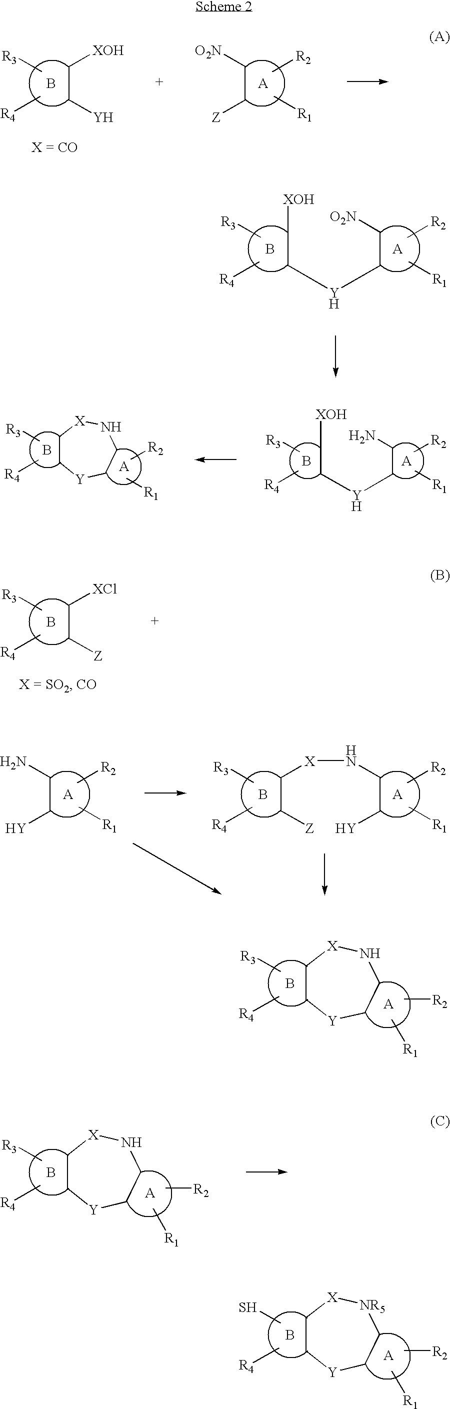

Some non-limiting examples of the present invention are described hereinafter:

### EXAMPLE 1

### Synthesis as Described in Scheme 2(A) and (C)

### 6-(11-Oxo-5,11-dihydro-dibenzo[b,e][1,4]diazepin-10-yl)-hexanoic acid hydroxyamide

Step 1 Anthranilic acid (10 g, 72.20 mmols) was combined with amyl alcohol (100 mls) and the mixture heated with stirring in an oil bath to 140° C. During the heating to this temperature, o-Bromo nitrobenzene (12.89 g, 64.40 mmols) was added followed by potassium carbonate (9 g, 65 mmols) and finally copper powder (0.4 g, 6.29×10-3 mols). After heating the mixture for less than 30 minutes at 140° C., a solid mass precipitated out making the mixture unstirrable. The solid mass was kept at this temperature for another 3 hours and then cooled to room temperature. The solid mass was transferred to a sintered glass funnel with help of diethyl ether (100 mls) to break up the solid mass. The solid was washed with further ether (3×100 mls) and dried by suction. The brick red solid was then dissolved in water (ca. 500 mls) and the resulting red solution filtered off from the catalyst. The filtrate was transferred back to a 1 L beaker and acidified with conc. HCl (50 mls). The resulting bright orange precipitate of the product was filtered off and dried by suction overnight. Yield 15.82 g (96%) of the coupling product.

HPLC (A)=4.03′; MS: [Ices+] MH+259.0

Step 2: The above obtained intermediate (16.46 g, 63.53 mmols) was combined with abs.ethanol (500 mls) and the mixture heated 78° C. Sodium dithionite (52 g, ca.85%, techn.grade, 253.99 mmols, 4 mole equivalents) was dissolved in water (230 mls) and added dropwise to the hot ethanolic solution of the substrate. A further aliquot of ethanol (100 mls) was then added to re-dissolve any remaining substrate and the final mixture was kept at 78° C. for 1 hr. After cooling back to room temperature, the mixture was filtered off from insoluble inorganic material which was washed with ethanol (2×150 mls). The combined filtrates were filtered again to remove further precipitated inorganic material. The operation was repeated once more with washing of the combined insoluble fractions with further ethanol (300 mls) and filtering a third time the combined filtrate to remove any further precipitated inorganic material. The final combined filtrate was stripped of ethanol under reduced pressure to give a slurry of the desired product which was taken up in water (140 mls). This slurry of the product was finally filtered off to give after drying by suction 11.06 g (76% yield) of the desired amine as a mustard yellow solid.

HPLC: t=2.85′.; MS[Ices+] MH+=229.0

Step 3 The 2-(2-Amino-phenylamino)-benzoic acid (2.50 g, 10.96 mmols) was suspended in acetonitrile (200 mls) and HOBt (4.40 g, 32.90 mmols) was added. After stirring for 10 minutes, EDC.HCl (3.10 g, 16.12 mmols) was added where it was noticed on addition of the coupling reagent that there was an intensification in the colour of the reaction mixture to a golden yellow together with a dissolution of the suspension. The mixture left to stir for 3 hrs. after which the acetonitrile was removed under reduced pressure. To the residue was added ethyl acetate (200 mls) followed by 10% aq. citric acid solution (100 mls). The two phases were vigorously agitated together in the reaction flask and then separated. The aqueous fraction was extracted with further ethyl acetate (200 mls). The combined ethyl acetate extracts were washed with saturated sodium bicarbonate solution (200 mls) and dried over sodium sulphate. Removal of solvent under reduced pressure gave 2.12 g (92% yield) of 5,10-Dihydro-dibenzo[b,e][1,4]diazepin-11-one as a yellow solid.

HPLC (A): 3.09′; MS[Ices+] MH+=211.3

Step 4: N-alkylation of 5,10-Dihydro-dibenzo[b,e][1,4]diazepin-11-one (500 mg, 2.37 mmols) with excess NaH (60% disperion in mineral oil) and Methyl 6-bromohexanoate (0.496 g, 2.37 mmols) in DMF at room temperature for 36 hrs (55% conversion to product) then with addition of further portions of sodium hydride (43 mg then 16 mg), gave according to analytical HPLC of the isolated crude product ca.89% conversion of the precursor to the desired N-hexyl carboxylate derivative. The product was isolated and treated with methanol (10 mls)/thionyl chloride (0.5 mls) to methylate the carboxylic acid side product which formed during the N-alkylation step. This gave on isolation 790 mg (98.5% yield) of the desired dibenzo diazepinyl methyl hexanoate ester derivative as a dark brown oil.

The above obtained intermediate was used directly for the conversion of the methyl ester to the hydroxamide by treatment of a methanolic solution of the substrate with hydroxylamine (prepared in-situ by liberation of the hydroxylamine hydrochloride with freshly prepared sodium methoxide in dry methanol). Yield: 105 mg (53%) of the desired hydroxamic acid.

The final product was purified further by preparative HPLC by dissolving in MeCN/H2O+0.1% TFA (1/1, v/v, 5 mls) and injecting in 2×2.50 ml aliquots directly onto the Shimadzu™ preparative HPLC system using the column Symmetry™ (C18.7 mm, 300 Å, 19×300 mm) and eluting according to the method H2O+0.1% TFA/MeCN+0.1% TFA, 70/30→10/90 in 60′, φ=20 ml/min, λ=220, 254 nm. Fraction volumes: 10 mls. Observed elution time for the above product 22.39-25.76′.

This gave after collection and lyophilization of the fractions, 84.70 mg of 6-(11-Oxo-5,11-dihydro-dibenzo[b,e][1,4]diazepin-10-yl)-hexanoic acid hydroxyamide (>95% titre by HPLC).

HPLC (A): 2.97′; MS[Ices+] MH+=340.2

¹H-NMR (DMSO-d6, 600 MHz) δ: 10.28 (1H, s)-8.94 (1H, bs)-7.79 (1H, s)-7.59 (1H, dd)-7.35-7.29 (2H, m)-7.14 (1H, m)-7.09-7.04 (3H, m)-6.94 (1H, t)-3.96 (2H, t)-2.19 (mc, t)-1.87 (2H, t)-1.47 (2H, m)-1.42 (2H, m)-1.23 (2H, m).

The following products were prepared in a similar manner to the aforementioned scheme using suitable commercial reagents, but with modifications well known to the expert of the art.

### EXAMPLE 2

### 6-(11-Oxo-11H-dibenzo[b,f][1,4]thiazepin-10-yl)-hexanoic acid hydroxyamide

HPLC (A): 3.38′; MS[Ices+] MH+=357.1

¹H-NMR (DMSO-d6, 600 MHz) δ: 10.28 (1H, s)-9.69 (mc, s)-7.64 (1H, dd)-7.61-7.58 (2H, m)-7.49 (1H, m)-7.42 (1H, t)-7.38 (2H, m)-7.20 (1H, td)-4.56 (1H, m)-3.62 (1H, m)-2.20 (mc, t)-1.88 (2H, t)-1.54-1.37 (4H, m)-1.33-1.22 (2H, m).

### EXAMPLE 3

### 6-(8-Methoxy-11-oxo-5,11-dihydro-dibenzo[b,e][1,4]diazepin-10-yl)-hexanoic acid hydroxyamide

HPLC (A): 3.12; MS[Ices+] MH+=370.1

¹H-NMR (DMSO-d6, 600 MHz) δ: 10.28 (1H, s)-9.68 (mc, s)-8.94-8.50 (1H, bs)-7.55 (2H, m)-7.29 (1H, t)-7.05 (1H, d)-7.01 (1H, d)-6.92 (1H, t)-6.90 (1H, d)-6.69 (1H, dd)-3.99 (2H, t)-2.20 (mc, bs)-1.88 (2H, t)-1.48 (2H, m)-1.43 (2H, m)-1.25 (2H, m).

### EXAMPLE 4

### 6-(8-Methoxy-11-oxo-11H-dibenzo[b,f][1,4]thiazepin-10-yl)-hexanoic acid hydroxyamide

HPLC (A): 3.32′-(B) 11.52′; MS[Ices+] MH+=387.0

¹H-NMR (DMSO-d6, 600 MHz) δ: 10.29 (1H, s)-9.70 (mc, s)-8.95 (mc, s)-8.62 (1H, s)-7.56 (1H, m)-7.51 (1H, d)-7.46 (1H, m)-7.37 (2H, m)-7.16 (1H, d)-6.78 (1H, dd)-4.57 (1H, m)-3.75 (3H, s)-3.65 (1H, m)-2.20 (mc, t)-1.89 (1H, t)-1.57-1.38 (1H, m)-1.29 (2H, m).

### EXAMPLE 5

### 6-(8-Chloro-11-oxo-5,11-dihydro-dibenzo[b,e][1,4]diazepin-10-yl)-hexanoic acid hydroxyamide

HPLC (A): 3.49′; MS[Ices+] MH+=374.1

NMR¹H-NMR (DMSO-d6, 600 MHz) δ: 10.28 (1H, s)-9.68 (mc, s)-8.93-8.59 (1H, bs)-7.91 (1H, s)-7.60 (1H, dd)-7.45 (1H, s)-7.33 (1H, t)-7.15 (2H, m)-7.04 (1H, d)-6.97 (1H, t)-3.99 (2H, t)-2.19 (mc, bs)-1.87 (2H, t)-1.44 (4H, m)-1.23 (2H, m).

### EXAMPLE 6

### 6-(8-Chloro-11-oxo-11H-dibenzo[b,f][1,4]thiazepin-10-yl)-hexanoic acid hydroxyamide

HPLC (A): 3.58′; MS[Ices+] MH+=391.1

¹H-NMR (DMSO-d6, 600 MHz) δ: 10.29 (1H, s)-9.69 (mc, bs)-8.94 (mc, bs)-8.61 (1H, bs)-7.76 (1H, d)-7.65 (1H, d)-7.59 (1H, m)-7.49 (1H, m)-7.40 (2H, m)-7.27 (1H, dd)-4.59 (1H, m)-3.63 (1H, m)-2.20 (mc, t)-1.89 (1H, t)-1.53-1.38 (4H, m)-1.28 (2H, m).

### EXAMPLE 7

### 6-(8-Methyl-11-oxo-5,11-dihydro-dibenzo[b,e][1,4]diazepin-10-yl)-hexanoic acid hydroxyamide

HPLC (A): 3.25′; MS[Ices+] MH+=354.2

¹H-NMR (DMSO-d6, 600 MHz) δ: 10.28 (1H, s)-9.69 (mc, bs)-8.94-8.54 (1H, bs)-7.65 (1H, s)-7.57 (1H, dd)-7.29 (1H, td)-7.15 (1H, s)-7.01 (2H, m)-6.92 (1H, t)-6.88 (1H, d)-3.96 (2H, t)-2.24 (3H, s)-1.88 (2H, t)-1.47 (2H, m)-1.43 (2H, m)-1.24 (2H, m).

Whenever necessary, the tricyclic skeleton is further processed before proceeding to the introduction of the pendant containing hydroxamic acid, in each case by means of reactions and methods known to the expert of the art. One of the most important of said processes is given by way of non-limiting example.

### EXAMPLE 8

### 6-(5,5,11-Trioxo-5,11-dihydro-5λ⁶-dibenzo[b,f][1,4]thiazepin-10-yl)-hexanoic acid hydroxyamide

The 6-(5,5,11-Trioxo-5,11-dihydro-56-dibenzo[b, f][1,4]thiazepin-10-yl)-hexanoic acid methyl ester (500 mg, 1.41 mmols), obtained as described in example 1, was dissolved in methanol (32 mls) and the solution treated with Oxone™ (0.97 g, 2.83 mmols) dissolved in water (16 mls). The mixture was stirred initially for 48 hours at room temperature with addition of another equivalent of the oxidizing agent (0.40 g) after 24 hours. However the reaction as indicated by analytical HPLC stopped mostly at sulphoxide (t=3.90′) stage with only 28% conversion further onto the sulphone product (t=4.15′). The mixture was then heated at 50° C. with addition of further oxone (0.40 g) after 7 hrs and the reaction continued overnight at the same temperature. The heating of the reaction was continued the following day with addition of further portions of oxone (2×0.40 g), then interrupted over the weekend period. The heating of the reaction mixture at 50° C. was then continued again for another 24 hours until 94% conversion of the sulphoxide to the sulphone was reached. The mixture was worked up by addition of water and removal of methanol under reduced pressure. The product was extracted with ethyl acetate (2×50 mls) and the combined organic extracts dried over sodium sulphate. Removal of solvent under reduced pressure gave 480 mg of a pale yellow oil. This material was treated with methanol (50 mls) and 4N HCl in dioxane (10 mls) and the solution stirred for 3 hours at room temperature. This converted the acid by-product present in the original reaction mixture (t=3.53′) back to the desired methyl ester product. The mixture was stripped of methanol under reduced pressure, the residue taken up in ethyl acetate (50 mls) and the solution washed with water (50 mls). The organic fraction was dried over sodium sulphate and solvent removed under reduced pressure to give 0.462 g (85% yield) of the desired product as a yellow oil which rapidly turned on standing to a waxy solid.

HPLC (A): 4.16′; MS[Ices+] MH+=388.1

Step 2: The sulphone intermediate (462 mg, 1.19 mmols) was dissolved in methanol (35 mls) and to the solution was added hydroxylamine hydrochloride (858 mg, 12.35 mmols). The solution was cooled to 0° C. in an ice-water bath and then treated with freshly prepared sodium methoxide (770 mg sodium, 33.50 mmols, in 15 mls of dry methanol). After stirring for 10 minutes the ice-bath was removed and the reaction continued for another 3 hours at room temperature. The reaction was then quenched by addition of water (25 mls) and the methanol removed by evaporation under reduced pressure. The aqueous residue was diluted with further water and neutralized by addition of 1M aq.HCl (50 mls). The precipitated product extracted with ethyl acetate (2×50 mls) and the combined extract washed with water (25 mls). Drying over sodium sulphate and removal of solvent under reduced pressure gave 355 mg of the crude hydroxamic acid product. A third extraction of the aqueous washings with ethyl acetate increased the amount of product obtained to 386 mg (83% yield).

HPLC (A): 3.06′; MS[Ices+] MH+=389.1

¹H-NMR (DMSO-d6, 600 MHz) δ: 10.30 (1H, s)-10.07 (mc, s)-8.95-8.57 (1H, bs)-7.95 (1H, dd)-7.86-7.82 (3H, m)-7.79 (1H, td)-7.76 (1H, t)-7.72 (1H, td)-7.49 (1H, t)-4.49 (1H, m)-3.80 (1H, m)-2.22 (mc, t)-1.90 (2H, t)-1.65 (1H, m)-1.51 (1H, m)-1.47 (2H, m)-1.26 (2H, m).

The following products were obtained in a similar manner or by other known synthesis processes.

### EXAMPLE 9

### 6-(8-Methoxy-5,5,11-trioxo-5,11-dihydro-5λ⁶-dibenzo[b,f][1,4]thiazepin-10-yl)-hexanoic acid hydroxyamide

HPLC (A): 3.04-10.37 (B); MS[Ices+] MH+=419.0

¹H-NMR (DMSO-d6, 600 MHz) δ: 10.31 (1H, s)-9.71 (mc, s)-8.96-8.59 (1H, bs)-7.84 (1H, d)-7.80 (2H, m)-7.76 (1H, t)-7.70 (1H, t)-7.32 (1H, d)-7.03 (1H, dd)-4.52 (1H, m)-3.79 (1H, m)-2.21 (mc, t)-1.91 (1H, t)-1.63 (1H, m)-1.51 (1H, m)-1.47 (2H, m)-1.27 (2H, m).

### EXAMPLE 10

### 6-(8-Chloro-5,5,11-trioxo-5,11-dihydro-5λ⁶-dibenzo[b,f][1,4]thiazepin-10-yl)-hexanoic acid hydroxyamide

HPLC (A): 3.26′; MS[Ices+] MH+=422.9

¹H-NMR (DMSO-d6, 600 MHz) δ: 10.31 (1H, s)-9.70 (mc, s)-8.95-8.63 (1H, bs)-8.00 (1H, d)-7.94 (1H, d)-7.86-7.80 (3H, m)-7.74 (1H, td)-7.57 (1H, dd)-4.54 (1H, m)-3.81 (1H, m)-2.23 (mc, m)-1.91 (1H, t)-1.62 (1H, m)-1.50 (1H, m)-1.47 (2H, m)-1.26 (2H, m).

### EXAMPLE 11

### 6-(8-Methoxy-5,11-dioxo-5,11-dihydro-5λ⁴-dibenzo[b,f][1,4]thiazepin-10-yl)-hexanoic acid hydroxyamide

HPLC (A): 2.8′; MS[Ices+] MH+=403.0

¹H-NMR (DMSO-d6, 600 MHz) δ: 10.30 (1H, s)-9.70 (mc, s)-8.95 (mc, s)-8.61 (1H, m)-7.69 (2H, t)-7.62 (1H, d)-7.55 (1H, tt)-7.49 (1H, d)-7.24 (1H, d)-7.05 (1H, d), 4.57 (1H, dt), 3.78 (3H, s)-3.67 (1H, m)-2.23 (mc, t)-1.91 (2H, t)-1.68-1.42 (4H, m)-1.29 (2H, m).

### EXAMPLE 12

### 6-(11-Oxo-11H-dibenzo[b,f][1,4]oxazepin-10-yl)-hexanoic acid hydroxyamide

Steps 1 &2: The dibenzo fused tricyclic azoxy intermediate, 2-nitrobenzo[b,f][11,4]oxazepin-11(10H)-one was prepared in two steps following the procedure described in the literature for the 7-Me substituted analogue reported by Klunder et al., J. Med. Chem., 1992, 35, 1887-1897. The first step involved the coupling of 2-chloro-5-nitrobenzoyl chloride with 2-aminophenol in THF in the presence of diisopropyl ethylamine with stirring at room temperature for 48 hrs. This gave the carboxamide intermediate in 92% yield.

Analytical HPLC (A) t=3.58′.; MS[Ices+] MH+=293.0

In the next step, the carboxamide intermediate was then suspended in water and treated with 2N aq. sodium hydroxide. Refluxing for a total of 10 hrs gave the closed-ring product as 85% yield after filtration of the solid material and drying by suction.

HPLC=3.66′.: MS[Ices+] MH+=257.2

Step 3: 2-nitrobenzo[b,f][1,4]oxazepin-11(10H)-one (2.00 g, 7.81 mmols) was suspended in water and abs. ethanol (25 mls+25 mls) and the suspension treated with elemental iron (0.36 g, 6.42 mmols) and iron (III) chloride (65 mg, 0.4 mmols). The suspension was refluxed for a total of 2.5 hrs. A further portion of iron (0.33 g) was added at 30 minutes and then again at 1 hour to the refluxing mixture. The mixture was then poured into excess ethanol and filtered off from the iron residues. The filtrate was stripped of ethanol under reduced pressure and the residue taken up in an excess volume of water. The product was filtered off and dried by suction. This gave 1.66 g (94% yield) of the amine as a light brick coloured solid.

HPLC (A)=2.19′; MS[Ices+] MH+=227.2

Step 4: DMF (15 mls) was heated in an oil bath to 50° C. and to this was added t-Butyl nitrite (0.98 mls, 7.47 mmols). The amine (1 g, 3.90 mmols) in DMF (10 mls) was added dropwise to the solution of t-Butyl nitrite at such a rate that the internal temperature did not exceed 50° C. After the addition of the substrate was completed, the mixture was kept at the same temperature for another 40 minutes. The mixture was cooled to room temperature and filtered through a sintered glass funnel. The filtrate was added dropwise to a mixture of water/conc.HCl (30 ml+30 ml) whereupon the product precipitated out. Further water (140 mls) was added and the mixture left to stir for 1 hr. The product was filtered off by suction and dried. Further product was obtained by extraction of the aqueous filtrate with ethyl acetate (2×50 mls). The ethyl acetate fraction was dried over sodium sulphate and solvent removed under reduced pressure to give solid residue which was treated with petroleum ether (40-60), the solid filtered off by suction and combined with the first crop of product. Further washing with petroleum ether of the combined crop of product and drying by suction gave 0.68 g (73% yield) of the dibenzo-oxazepinone as a buff coloured solid.

HPLC (A)=3.45′; MS[Ices+] MH+=212.2

Step 5: The tricycle was transformed into the final product using the methods already described in the preceding examples

HPLC (A)=3.25′; MS[Ices+] MH+=341.1

¹H-NMR (DMSO-d6, 600 MHz) δ: 10.29 (1H, s)-9.69 (mc, s)-8.95-8.54 (1, bs)-7.70 (1H, dd)-7.55 (1H, td)-7.54 (1H, td)-7.38 (1H, dd)-7.33 (1H, d)-7.30-7.26 (2H, m)-7.22 (1H, td)-4.09 (2H, bs)-2.21 (mc, t)-1.89 (2H, t)-1.56 (2H, m)-1.46 (2H, m)-1.25 (2H, m).

The following products were obtained in a similar manner:

### EXAMPLE 13

### 6-(8-Methoxy-11-oxo-11H-dibenzo[b,f][1,4]oxazepin-10-yl)-hexanoic acid hydroxyamide

HPLC (A)=3.22; MS[Ices+] MH+=371.1

¹H-NMR (DMSO-d6, 600 MHz) δ: 10.30 (1H, s)-9.70 (mc, s)-8.97-8.23 (1H, bs)-7.80 (1H, s)-7.65 (1H, d)-7.43 (2H, m)-6.89 (1H, m)-6.86 (1H, d)-6.83 (1H, dd)-6.61 (1H, d)-6.02 (3H, bs)-3.11 (2H, bs)-2.21 (mc, t)-1.89 (2H, t)-1.42 (4H, m)-1.27 (2H, bs).

### EXAMPLE 14

### 6-(8-Chloro-11-oxo-11H-dibenzo[b,f][1,4]oxazepin-10-yl)-hexanoic acid hydroxyamide

HPLC (A)=3.49′; MS[Ices+] MH+=375.1

¹H-NMR (DMSO-d6, 600 MHz) δ: 10.28 (1H, s)-9.69 (mc, s)-8.94 (mc, s)-8.61 (1H, s)-7.71 (1H, dd)-7.66 (1H, dd)-7.57 (1H, ddd)-7.42 (1H, d)-7.35 (1H, d)-7.32-7.28 (2H, m)-4.11 (2H, bs)-2.21 (mc, t)-1.89 (2H, t)-1.53 (2H, m)-1.46 (2H, m)-1.24 (2H, m).

### EXAMPLE 15

### 7-(11-Oxo-11H-dibenzo[b,f][1,4]oxazepin-10-yl)-heptanoic acid hydroxyamide

HPLC (B)=11.57′; MS[Ices+] MH+=355.1

¹H-NMR (DMSO-d6, 600 MHz) δ: 10.29 (1H, s)-9.69 (mc, s)-8.95 (mc, s)-8.62 (1H, s)-7.70 (1H, dd)-7.56-7.52 (2H, m)-7.38 (1H, dd)-7.33 (1H, dd)-7.28 (2H, qd)-7.22 (1H, td)-4.10 (2H, bs)-2.21 (mc, t)-1.89 (1H, t)-1.55 (2H, m)-1.41 (2H, m)-1.26 (2H, m)-1.20 (2H, m).

### EXAMPLE 16 SCHEME 2 (A) (C)

### 6-(5-Oxo-5,11-dihydro-benzo[b]pyrido[2,3-e][1,4]diazepin-6-yl)-hexanoic acid hydroxyamide

Step 1: A suspension obtained with 108 mg (1 eq., 1 mmol) of o-phenylenediamine and 157 mg (1 eq., 1 mmol) of 2-chloro-nicotinic acid in diethylene glycol monomethyl ether, are heated to 150° C. for 6 hours. The suspension is allowed to return to ambient temperature and then the entirety is poured onto water cooled to 0° C. It is stirred for 20 minutes then the brownish precipitate formed is filtered off through a porous septum and left to dry in the air on filter paper. 115 g of a solid are thus obtained (Yield 54%).

HPLC (B)=7.1′; MS[Ices+] MH+=212.2

The tricycle obtained is then transformed into the final product using the already described procedure.

HPLC (B)=7.73′; MS[Ices+] MH+=341.0

¹H-NMR (DMSO-d6, 600 MHz) δ: 10.27 (1H, s)-9.68 (mc, s)-8.59 (1H, s)-8.26 (1H, dd)-8.01 (1H, dd)-7.37 (1H, m)-7.26 (1H, m)-7.12 (2H, m)-7.02 (1H, dd)-3.98 (2H, t)-2.19 (mc, t)-1.87 (2H, t)-1.45 (2H, m)-1.41 (2H, m)-1.22 (2H, m).

The following were obtained in exactly the same manner:

### EXAMPLE 17

### 6-(6,7-Dichloro-10-oxo-4H,10H-2-thia-4,9-diaza-benzo[f]azulen-9-yl)-hexanoic acid hydroxyamide

HPLC (A)=3.52′; MS[Ices+] MH+=314.1

¹H-NMR (DMSO-d6, 600 MHz) δ: 10.29 (1H, s)-9.70 (mc, s)-8.95 (mc, s)-8.63 (1H, bs)-8.25 (1H, s)-8.04 (1H, d)-7.64 (1H, s)-7.31 (1H, s)-6.65 (1H, d)-8.97 (2H, t)-2.18 (mc, t)-1.87 (2H, t)-1.41 (4H, m)-1.20 (2H, m).

### EXAMPLE 18

### 6-(8-Methoxy-5-oxo-5,11-dihydro-benzo[b]pyrido[2,3-e][1,4]diazepin-6-yl)-hexanoic acid hydroxyamide

HPLC (B)=7.98 (B); MS[Ices+] MH+=371.1

¹H-NMR (DMSO-d6, 600 MHz) δ: 10.27 (1H, s)-9.68 (mc, s)-8.93 (mc, s)-8.61 (1H, s)-8.36 (1H, s) 8.23 (1H, dd)-7.98 (1H, dd)-7.16 (1H, d)-6.99 (1H, dd)-6.92 (1H, d)-6.74 (1H, dd)-4.00 (2H, t)-3.72 (3H, s)-2.19 (mc, t)-1.87 (2H, t)-1.49-1.40 (4H, m)-1.23 (2H, m).

### EXAMPLE 19

### 6-(8,9-Dimethyl-5-oxo-5,11-dihydro-benzo[b]pyrido[2,3-e][1,4]diazepin-6-yl)-hexanoic acid hydroxyamide

HPLC (B)=7.01 (B); MS[Ices+] MH+=369.1

¹H-NMR (DMSO-d6, 600 MHz) δ: 10.27 (1H, s)-9.68 (mc, s)-8.93 (mc, s)-8.61 (1H, s)-8.32 (1H, s)-8.22 (1H, dd)-7.97 (1H, dd)-7.13 (1H, s)-6.99-6.97 (2H, m)-3.95 (1H, t)-2.16 (3H, s)-2.13 (3H, s)-1.87 (2H, t)-1.43 (4H, m)-1.22 (2H, m).

### EXAMPLE 20 (B) (C)

### 6-(8-Dimethylamino-10,10-dioxo-5,10-dihydro-10λ⁶-thia-5,11-diaza-dibenzo[a,d]cyclohepten-11-yl)-hexanoic acid hydroxyamide

Step 1: 1-chloro-4-nitrobenzene (6.93 g, 44 mmols) is added to a flask containing chlorosulphonic acid (20 ml) and heated to 120° C. for 16 hours. After decomposing an aliquot of the reaction mixture and extracting with dichloromethane, GC-Mass analysis is undertaken, showing 74% of product and 14% of unreacted initial substance. The reaction is then stopped by pouring it carefully onto ice, extracting with dichloromethane, washing with brine, drying on a phase separator and evaporating to dryness.

9.17 g of a semi-solid product is obtained and used as such for the subsequent synthesis.

Step 2: Synthesis of 3-Nitro-6,11-dihydro-dibenzo[c,f][1,2]thiazepine 5,5-dioxide Orthophenylenediamine (44.4 mmols, 4.8 g) is suspended in pyridine (20 ml) then sulphur chloride is slowly added to this suspension, finally resuspending in pyridine to remove it from the flask. As the reaction is exothermic it is cooled in a water bath. After addition is complete the suspension is refluxed for 1.5 h. HPLC monitoring shows the disappearance of the sulphur chloride and formation of the product. The reaction mixture is evaporated to dryness and the residue is treated with 1N HCl to pH 1, extracted with ethyl acetate, washed with brine and dried over MgSO₄. By evaporation of the solvent a residue is obtained which solidifies on treatment with ethyl ether and is then filtered off and washed with ether. 4.35 g of 3-nitro-6,11-dihydro-dibenzo[c,f][1,2]thiazepine 5,5-dioxide are obtained as a yellow solid.

HPLC (A)=3.4′; MS[Ices+] MH+=291.4

Step 3: The solid thus obtained (6 mmols, 1.746 g) is dissolved in methanol (50 ml) and treated with a methanolic solution of sodium methoxide (6 mmols: 36 ml of solution containing 385 mg of sodium in 100 ml of methanol). The solution obtained is then dried and evaporated to dryness by mechanical pump to obtain the corresponding sodium salt as a solid. This compound is dissolved in DMF (30 ml), methyl 6-bromo hexanoate (6 mmols, 1.45 g) in DMF (10 ml) are added and the mixture heated to 100° C. for 3 h until the reaction is complete, monitored by HPLC. The reaction mixture is evaporated under vacuum by mechanical pump, the residue is treated with brine and extracted with ethyl acetate, dried and evaporated to dryness to obtain the product in a quantitative yield.

HPLC (A)=4.45′; MS[Ices+] MH+=419.8

Step 4: The alkylated intermediate compound (4.5 mmols, 1.9 g) is dissolved in hot glacial acetic acid (80 ml) and the first portion of iron reduced by hydrogen (2.5 g, 45 mmols, divided into 4 portions) is added. The mixture is refluxed, maintaining at reflux for 1.5 h; in the first hour the remaining 3 portions of iron are added. After about 1 h at reflux the reaction mixture appears as a beige coloured milky suspension. At the end of the reaction the reaction mixture is cooled to 60° C. and filtered through a septum, washing the precipitate with acetic acid. The filtrate is evaporated to dryness and the residue treated with water, extracted with DCM, washed with 5% NaHCO₃ and dried. After evaporating the solvent the methyl ester is obtained as a solid (1.57 g).

The solid ester is suspended in methanol (30 ml), treated with 1N NaOH (8 mmols, 8 ml) and held for 1 hour at reflux, observing the disappearance of the ester and formation of the acid by HPLC. The methanol is evaporated from the reaction mixture under vacuum, the mixture is diluted with water and ethyl acetate (50 ml), the impurities are extracted and the residual aqueous solution is acidified with 1N HCl. The solid that separates is extracted with ethyl acetate which is dried and evaporated thus obtaining a solid of 1.29 g, yield 85.8%.

HPLC (A)=3.19′; MS[Ices+] MH+=418.0

The 6-(8-Acetylamino-10,10-dioxo-5,10-dihydro-10λ⁶-thia-5,11-diaza-dibenzo[a,d]cyclohepten-11-yl)-hexanoic acid thus obtained (387 mg, 0.93 mmols) is treated with 95° ethanol (10 ml) and conc. HCl in water (2 ml) and held at reflux for 1 hour, monitoring by HPLC the disappearance of the reagent and formation of 30% acid and 70% ethyl ester. The reaction mixture is concentrated by rotavapor and the residue treated with brine. The mixture is extracted with ethyl acetate which, after drying and evaporating to dryness, provides a solid of 290 mg that is used in the crude form in the next reaction.

The previously obtained crude mixture (290 mg) is dissolved in methanol (8 ml) to which are added paraformaldehyde (105 mg, 3.5 mmols), acetic acid (0.15 ml, 2.5 mmols) and NaCNBH₃ (126 mg, 2 mmols). The mixture is stirred for 48 h at ambient temperature achieving total transformation into demethylated derivatives. The reaction mixture is acidified with 1N HCl and after ½ h is alkalinised with 1N NaOH (8 ml) and held at reflux for ½ h, to obtain the acid derivative alone. After cooling, the product is acidified with 1N HCl and extracted with ethyl acetate, then, after washing with brine and drying, is evaporated to provide the 6-(8-Dimethylamino-10,10-dioxo-5,10-dihydro-10λ⁶-thia-5,11-diaza-dibenzo[a,d]cyclohepten-11-yl)-hexanoic acid as a solid of 232 mg, yield 62%.

HPLC (A)=2.94′; MS[Ices+] MH+=404.1

The intermediate (232 mg, 0.58 mmols) is dissolved in DMF (10 ml) and Et₃N (1.1 mmols, 0.16 ml) is added at −10° C. Ethyl chloroformate (1 mmol, 0.1 ml) is added drop-wise and the mixture is maintained between −10 and 0° C. for 1 hour. At the end of this period, this suspension is added in total to a mixture of NH₂OH*HCl (2.8 mmols, 200 mg) in DMF (3 ml) to which Et₃N (2.9 mmols, 0.4 ml) was added. The resulting reaction mixture is maintained for 2 hours while stirring. Formation of the hydroxamate is observed by HPLC. The reaction mixture is dried by mechanical pump, diluted with brine and extracted with ethyl acetate (twice). The extract is dried and by evaporating the solvent a crude oil is obtained which is purified by preparative Schimatzu HPLC (3 passages) with a Symmetry Prep C18 19×300 mm column with an eluent mixture formed of 80% water and 20% acetonitrile (both containing 0.1% TFA), the CH3CN increasing with linear gradient by 0.5% per minute. The pure chromatographic fractions are collected and lyophilised.

A white lyophilised solid of 150 mg, yield 48.5%, is obtained.

HPLC (A)=2.5; MS[Ices+] MH+=419.1

¹H-NMR (DMSO-d6, 600 MHz) δ: 10.29 (1H, s)-9.70 (mc, s)-9.09 (1H, s)-7.26-7.24 (2H, m)-7.22 (1H, m)-7.14 (3H, m)-6.91 (1H, t)-2.98 (2H, bs)-2.93 (3H, m)-2.20 (mc, t)-1.89 (2H, t)-1.40 (4H, m)-1.25 (2H, m).

The following products were formed in a similar manner.

### EXAMPLE 21

### 6-(3-Methoxy-10,10-dioxo-5,10-dihydro-10λ⁶-thia-5,11-diaza-dibenzo[a,d]cyclohepten-11-yl)-hexanoic acid hydroxyamide

HPLC (A)=3.11; MS[Ices+] MH+=462.1

¹H-NMR (DMSO-d6, 600 MHz) δ: 10.29 (1H, s)-9.70 (mc, s)-9.49 (mc, s)-9.30 (1H, s)-8.95 (mc, s)-8.62 (1H, s)-7.68 (1H, dd)-7.46 (1H, td)-7.26 (1H, d)-7.08 (1H, d)-6.92 (1H, t)-6.74 (1H, d)-6.58 (1H, dd)-3.76 (3H, s)-2.95 (2H, bs)-2.20 (mc, t)-1.89 (2H, t)-1.44-1.37 (4H, m)-1.25 (2H, bs).

### EXAMPLE 22

### 6-(10,10-Dioxo-5,10-dihydro-10λ⁶-thia-5,11-diaza-dibenzo[a,d]cyclohepten-11-yl)-hexanoic acid hydroxyamide

HPLC (A)=3′; MS[Ices+] MH+=376.1

¹H-NMR (DMSO-d6, 600 MHz) δ: 10.28 (1H, s)-9.69 (mc, s)-9.35 (1H, s)-8.95 (mc, s)-7.69 (1H, dd)-7.47 (1H, td)-7.30 (2H, m)-7.19 (2H, m)-6.99 (1H, t)-6.91 (1H, t)-3.00 (2H, bs)-2.20 (mc, t)-1.88 (2H, t)-1.40 (4H, m)-1.25 (2H, m).

### EXAMPLE 23

### 6-(10,10-Dioxo-10H-5-oxa-10λ⁶-thia-11-aza-dibenzo[a,d]cyclohepten-11-yl)-hexanoic acid hydroxyamide

HPLC (A)=3.28; MS[Ices+] MH+=377.1

¹H-NMR (DMSO-d6, 600 MHz) δ: 10.30 (1H, s)-9.71 (mc, s)-8.95 (mc, s)-8.63 (1H, s)-7.80 (1H, dd)-7.68 (1H, td)-7.50-7.44 (4H, m)-7.39-7.34 (2H, m)-3.54 (2H, t)-2.21 (mc, t)-1.90 (2H, t)-1.44 (4H, m)-1.29 (2H, m).

### EXAMPLE 24

### 6-(8-Amino-10,10-dioxo-5,10-dihydro-10λ⁶-thia-5,11-diaza-dibenzo[a,d]cyclohepten-11-yl)-hexanoic acid hydroxyamide

HPLC (A)=2.38; MS[Ices+] MH+=391.1

¹H-NMR (DMSO-d6, 600 MHz) δ: 10.30 (1H, bs)-9.70 (mc, bs)-9.28 (1H, s)-8.97-8.26 (1H, bs)-7.41 (1H, s)-7.28 (1H, t)-7.25 (1H, d)-7.19-7.16 (3H, m)-6.96 (1H, t)-3.00 (2H, bs)-2.20 (mc, t)-1.89 (2H, t)-1.40 (4H, m)-1.24 (2H, m).

### EXAMPLE 25

### 6-(2-Fluoro-10,10-dioxo-5,10-dihydro-10λ⁶-thia-5,11-diaza-dibenzo[a,d]cyclohepten-11-yl)-hexanoic acid hydroxyamide

HPLC (A)=3.09; MS[Ices+] MH+=394.1

¹H-NMR (DMSO-d6, 600 MHz) δ: 10.28 (1H, s)-10.08 (mc, s)-9.69 (mc, s)-9.35 (1H, s)-8.93-8.50 (1H, bs)-7.68 (1H, dd)-7.47 (1H, td)-7.25 (1H, d)-7.21 (2H, m)-7.12 (1H, d)-6.91 (1H, t)-3.05 (2H, bs)-2.19 (mc, t)-1.88 (2H, t)-1.40 (4H, m)-1.23 (2H, m).

### EXAMPLE 26

### 6-(8-Dimethylamino-3-hydroxy-10,10-dioxo-5,10-dihydro-10λ⁶-thia-5,11-diaza-dibenzo[a,d]cyclohepten-11-yl)-hexanoic acid hydroxyamide

HPLC (A)=2.01; MS[Ices+] MH+=435.1

¹H-NMR (DMSO-d6, 600 MHz) δ: 10.29 (1H, s)-9.58 (1H, s)-8.88 (1H, s)-7.21 (1H, d)-7.18 (1H, bs)-7.09 (1H, bs)-6.91 (1H, d)-6.51 (1H, s)-6.33 (1H, d)-3.14-2.74 (2H, bs)-2.91 (3H, s)-2.21 (mc, t)-1.89 (2H, t)-1.42 (2H, m)-1.38 (2H, m)-1.25 (2H, bs).

### EXAMPLE 27

### 6-(8-Dimethylamino-3-methoxy-10,10-dioxo-5,10-dihydro-10λ⁶-thia-5,11-diaza-dibenzo[a,d]cyclohepten-11-yl)-hexanoic acid hydroxyamide

HPLC (A)=2.42; MS[Ices+] MH+=449.1

¹H-NMR (DMSO-d6, 600 MHz) δ: 10.30 (1H, s)-9.71 (mc, s)-8.89 (1H, s)-8.63 (1H, s)-7.16 (1H, d)-7.05 (1H, dd)-7.01 (1H, d)-6.90 (1H, d)-6.64 (1H, d)-6.47 (1H, d)-3.74 (3H, s)-3.03 (2H, bs)-2.85 (6H, s)-2.20 (mc, t)-1.89 (2H, t)-1.41 (2H, m)-1.37 (2H, m)-1.25 (2H, m).

### EXAMPLE 28

### 6-(7-Methyl-10,10-dioxo-5,10-dihydro-10λ⁶-thia-5,11-diaza-dibenzo[a,d]cyclohepten-11-yl)-hexanoic acid hydroxyamide

HPLC (A)=3.3′; MS[Ices+] MH+=390.1

¹H-NMR (DMSO-d6, 600 MHz) δ: 10.28 (1H, s)-9.69 (mc, s)-9.26 (1H, s)-8.94 (1H, bs)-7.57 (1H, d)-7.28 (1H, td)-7.18 (2H, m)-7.10 (1H, s)-6.97 (1H, td)-6.74 (1H, d)-2.96 (2H, bs)-2.30 (3H, s)-2.20 (mc, bs)-1.88 (2H, t)-1.43-1.36 (4H, m)-1.24 (2H, m).

### EXAMPLE 29

### 6-(2-Methoxy-10,10-dioxo-5,10-dihydro-10λ⁶-thia-5,11-diaza-dibenzo[a,d]cyclohepten-11-yl)-hexanoic acid hydroxyamide

HPLC (A)=3.18; MS[Ices+] MH+=406.1

¹H-NMR (DMSO-d6, 600 MHz) δ: 10.28 (1H, s)-9.69 (mc, s)-9.15 (1H, s)-7.65 (1H, d)-7.42 (1H, t)-7.21 (1H, d)-7.12 (1H, d)-6.95 (1H, dd)-6.85 (1H, t)-6.75 (1H, d)-3.75 (3H, s)-3.05 (2H, bs)-2.20 (mc, t)-1.89 (2H, t)-1.42 (4H, m)-1.25 (2H, m).

### EXAMPLE 30

### 6-(7-Methoxy-10,10-dioxo-5,10-dihydro-10λ⁶-thia-5,11-diaza-dibenzo[a,d]cyclohepten-11-yl)-hexanoic acid hydroxyamide

HPLC (A)=3.2′; MS[Ices+] MH+=406.1

¹H-NMR (DMSO-d6, 600 MHz) δ: 10.29 (1H, s)-9.70 (mc, s)-9.32 (1H, s)-8.94-8.47 (1H, bs)-7.60 (1H, d)-7.29 (1H, t)-7.17 (2H, m)-6.98 (1H, t)-6.82 (1H, d)-6.53 (1H, dd)-3.81 (1H, s)-2.96 (2H, bs)-2.20 (mc, t)-1.89 (2H, t)-1.44-1.37 (4H, m)-1.25 (2H, m).

### EXAMPLE 31

### 6-(11-Methyl-10,10-dioxo-10,11-dihydro-5H-10λ⁶-thia-5,11-diaza-dibenzo[a,d]cyclohepten-7-yloxy)-hexanoic acid hydroxyamide

HPLC (A)=3.43′; MS[Ices+] MH+=406.1

¹H-NMR (DMSO-d6, 600 MHz) δ: 10.34 (1H, s)-9.74 (mc, s)-9.26 (1H, s)-9.00 (mc, s)-8.65 (1H, bs)-7.58 (1H, d)-7.26 (1H, t)-7.24 (1H, d)-7.12 (1H, d)-6.96 (1H, d)-6.52 (1H, dd)-4.01 (2H, t)-2.83 (3H, s)-2.30 (mc, t)-1.98 (2H, t)-1.74 (2H, m)-1.57 (2H, m)-1.39 (2H, m).

### EXAMPLE 32

### 6-(4-Amino-10,10-dioxo-5,10-dihydro-10λ⁶-thia-5,11-diaza-dibenzo[a,d]cyclohepten-11-yl)-hexanoic acid hydroxyamide

HPLC (A)=3.28′; MS[Ices+] MH+=391.2

¹H-NMR (DMSO-d6, 600 MHz) δ: 10.30 (1H, s)-9.70 (mc, s)-8.97-8.23 (1H, bs)-7.80 (1H, s)-7.65 (1H, d)-7.43 (2H, m)-6.89 (1H, m)-6.86 (1H, d)-6.83 (1H, dd)-6.61 (1H, d)-6.02 (3H, bs)-3.11 (2H, bs)-2.21 (mc, t)-1.89 (2H, t)-1.42 (4H, m)-1.27 (2H, bs).

### EXAMPLE 33

### 6-(10-Oxo-4H,10H-2-thia-4,9-diaza-benzo[f]azulen-9-yl)-hexanoic acid hydroxyamide

Step 1: 1.1 g of metallic sodium previously cut into thin slices are added to 11 ml of methanol under vigorous agitation. The resulting solution is heated to reflux and 3.0 g of methyl 3-[(2-methoxy-2-oxoethyl)thio]propanoate are slowly added (about 10 minutes). The solution is refluxed again for 30 minutes then allowed to return to ambient temperature. The entirety is poured onto ice and water (about 100 ml) while stirring, then stirred for 30-40 minutes and acidified to pH 2 with conc. HCl. The waters are extracted 5 times with dichloromethane, the organic extracts are pooled and dried, then concentrated by rotavapor to obtain 1.7 g of an oil.

GC-MS analysis shows the presence of the other isomer (methyl tetrahydro-3-oxa-2-thiophenecarboxylate) at ca 3% (HPLC (A)=2.53′). The crude product is purified with a Flash Master Personal and a STRATA column pre-packed with silica (20 g) from phenomenex. The crude product is dissolved in dichloromethane:hexane=1:1, then dry loaded and eluted with dichloromethane:hexane=1:1.

1.12 g of a white solid are obtained. Yield: 54%.

HPLC (A)=2.61′

Step 2: 1,3,4,9-Tetrahydro-10H-thieno[3,4-b][1,5]benzodiazepin-10-one

A solution obtained by dissolving 1.12 g of methyl tetrahydro-4-oxa-3-thiophenecarboxylate and 0.76 g of o-phenylendiamine in 27 ml of anhydrous toluene is heated at reflux for 2.5 h using a Dean-Stark trap to remove the water. The solution is allowed to return to ambient temperature. An orange precipitate forms which is filtered through a porous septum and left in air to dry. 1.14 g of clean 1,3,4,9-tetrahydro-10H-thieno[3,4-b][1,5]benzodiazepin-10-one are thus obtained. Yield: 75%

HPLC (A)=2.43′

MS[Ices+] MH+=219.2

Step 3: 698 mg of N-chlorosuccinimide are added in portions to a mixture of 1.14 g of the thus obtained product in 11 ml of anhydrous pyridine under nitrogen while stirring such that the internal temperature of the reaction remains between 10 and 15° C. with the assistance of an ice and water bath. At the end of the addition the entirety is brought to 60° C. for 30 minutes and then brought to ambient temperature. The reaction mixture is poured onto 100 ml of water and ice and left for 20 minutes while stirring. The precipitate that forms is then filtered off through a porous septum then allowed to dry on filter paper for a few hours. 1.01 g of 4,9-dihydro-10H-thieno[3,4-b][1,5]benzodiazepin-10-one are obtained with a purity >95%. Yield: 90%

HPLC (A)=2.77′; MS[Ices+] MH+=217.2

The tricycle is transformed into the final product in a manner similar to that described.

HPLC (A)=2.82′; MS[Ices+] MH+=346.1

¹H-NMR (DMSO-d6, 600 MHz) δ: 10.27 (1H, s)-9.68 (mc, s)-8.94 (mc, s)-8.61 (1H, s)-7.96 (2H, d)-7.31 (1H, d)-7.08 (2H, m)-7.03 (1H, m)-6.60 (1H, d)-3.94 (2H, t)-2.18 (mc, t)-1.86 (2H, t)-1.46-1.38 (4H, m)-1.21 (2H, m).

The following are obtained in exactly the same manner:

### EXAMPLE 34

### 6-(6,7-Dichloro-10-oxo-4H,10H-2-thia-4,9-diaza-benzo[f]azulen-9-yl)-hexanoic acid hydroxyamide

HPLC (A)=3.52; MS[Ices+] MH+=314.1

NMR¹H-NMR (DMSO-d6, 600 MHz) δ: 10.29 (1H, s)-9.70 (mc, s)-8.95 (mc, s)-8.63 (1H, bs)-8.25 (1H, s)-8.04 (1H, d)-7.64 (1H, s)-7.31 (1H, s)-6.65 (1H, d)-8.97 (2H, t)-2.18 (mc, t)-1.87 (2H, t)-1.41 (4H, m)-1.20 (2H, m).

### EXAMPLE 35

### N-Hydroxy-4-[1-(11-oxo-10,11-dihydro-5H-dibenzo[b,e][1,4]diazepine-6-carbonyl)-piperidin-4-yl]-butyramide

HPLC (B)=7.66′; MS[Ices+] MH+=423.1

¹H-NMR (DMSO-d6, 600 MHz) δ: 10.32 (1H, s)-10.06 (1H, s)-9.73 (mc, s)-8.99 (mc, s)-8.65 (1H, s)-7.67 (1H, dd)-7.37 (1H, t)-7.06 (1H, d)-7.01-6.95 (3H, m)-6.90 (2H, t)-4.54 (1H, d)-3.37 (1H, d)-2.93 (1H, bs)-2.81 (1H, t)-2.25 (mc, t)-1.92 (2H, t)-1.76 (1H, m)-1.48 (4H, m)-1.18 (3H, bs)-0.84 (1H, bs).

HPLC Methods:

(A) Zorbax™ Column, SB-18, 3.5 mm, 100 Å (50×4.6 mm), H₂O+0.1% TFA/MeCN+0.1% TFA, from 95/5 to 5/95 in 6.5 min+1 min isocratic, φ=3 ml/min, λ=220, 254 nm

(B) Symmetry 300 Column, C-18, 5 micron (250×4.6 mm), H₂O+0.1% TFA/MeCN+0.1% TFA, from 85/15 to 5/95 in 20 min+4 min isocratic, φ=1 ml/min, λ=210 nm

### NMR Abbreviations:

mc=minor conformer
bs=broad signal
m=multiplet or overlapping multiplets

### Therapeutic Indications

The histone deacetylase inhibitors are a class of potential therapeutic or prophylactic agents for pathological states caused by abnormal gene expression, such as inflammatory disorders, diabetes, complications of diabetes, homozygotic thalassaemia, fibrosis, cirrhosis, acute promyelocytic leukaemia (APL), transplant rejection, auto-immune diseases, protozoan infections, tumors and the like. In particular they are emerging as a new class of drugs with anti-tumor activity. The connection between some tumorous pathologies, such as carcinoma of the mammary, colon and lung, and acetylation levels of nuclear chromatin has been described. Drugs able to modulate chromatin remodelling are able to inhibit tumor proliferation and could provide new instruments for treating tumor pathologies in the not too distant future. Much experimental evidence leads to the belief that the main application field of these drugs could be in combined therapies. The considerable tolerability that has emerged from the first clinical trials leads to the belief that this class of molecules lends itself to combined therapy with traditional drugs such as cytotoxic drugs, or with radiotherapy treatments or with the new generation antitumor agents. In particular, the present invention also provides combinations of compounds with histone deacetylase inhibitory activity of general formula (I) together with one or more chemotherapeutic compounds chosen from the group: conventional cytotoxic agents, demethylating agents, cyclin dependent kinase inhibitors, differentiating agents, signal transduction modulators, HSP-90 antagonists, proteasome inhibitors. Preferred compounds are compounds chosen from the following groups: the conventional cytotoxic agents: fludarabine, gemcitabine, decitabine, paclitaxel, carboplatin and Topo I/II inhibitors to include Etoposide, Irinotecan, Topotecan, T-128 and Anthracyclines such as Doxorubicin, Sabarubicin, Daunorubicin;

the demethylating agents (demethylation of DNA): 5-aza-2′-deoxycytidine (5-aza-dC), 5-azacytidine;
the cyclin dependent kinase inhibitors: Flavopiridol, olomoucin, roscovitin, purvalanol B, GW9499, GW5181, CGP60474, CGP74514, AG12286, AG12275, Staurosporine, UCN-01;
the differentiating agents: retinoic acid and derivatives (All Trans Retinoic Acid, ATRA), 13-cis retinoic acid (CRA), PMA (phorbol myristate acetate); the signal transduction modulators: TRAIL, imatinib mesylate, LY-294002, bortezomib;
the HSP-90 antagonists: geldanamycin and its analogues (17-AAG);
the proteasome inhibitors: lactacystine, MG132, bortezomib (Velcade™).

### Biological Activity

The activity of the compounds as histone deacetylase (HDAC) inhibitors was measured using an in vitro acetylation assay. The compounds were then evaluated as inhibitors of proliferation of human tumor cell cultures. The overall data obtained are given in the table.

### Deacetylase Activity on Nuclear Extract of Hela Cells (Human Cervical Cancer Cell)

The assay (Fluor de Lys™ kit, BioMol) is divided into two steps: in the first step the substrate which comprises an acetylated lysine residue is reacted with the nuclear extract (HeLa) containing the enzymatic activity in the presence and absence of inhibitors. In the second step a fluorogenic reagent is added which highlights the deacetylated residues. A reduction in fluorescence is obtained where there has been inhibition of the deacetylase activity. The result is finally expressed as percent inhibition relative to the control without inhibitor at a concentration of 1 μM.

### Evaluation of Cytotoxic Activity on Culture of Human Colon Carcinoma Cells HCT-116

Human colon carcinoma cells HCT-116 were seeded onto 96-well plates in RPMI1640 culture medium with added 10% FBS and 2 mM glutamine. 24 hours after seeding, the compounds at different concentrations are added. All the compounds are diluted in DMSO such that the final concentrations on the cultures is no greater than 0.5%. 72 hours after addition of the compounds, cell viability is measured by means of the dye Alamar Blue. The result is expressed as percent survival of the treated relative to the control, treated with carrier alone.

| EXAMPLE | % inhib at 0.1 μM | IC50 (HCT-116) |
|---|---|---|
| 8 | 73 | 0.105 |
| 9 | 83 | 0.04 |
| 10 | 77 | 0.03 |
| 11 | 73 | 0.05 |
| 12 | 66 | 0.64 |
| 13 | 73 | 0.31 |
| 14 | 65 | 0.2 |
| 15 | 62 | 0.2 |
| 17 | 40 | 0.8 |
| 20 | 40 | 0.39 |
| 21 | 57 | 0.29 |
| 23 | 48 | 0.19 |
| 25 | 42 | 1.5 |
| 26 | 66 | 1 |
| 27 | 63 | 0.4 |
| 29 | 54 | 0.9 |
| 30 | 65 | 0.39 |
| 32 | 47 | 2 |
| 34 | 40 | 0.8 |

In the same test, suberanilohydroxamic acid (SAHA), which was included as reference, demonstrated an inhibitory effect of 55% at 0.1 μM.

1. Compounds of general formula (I): 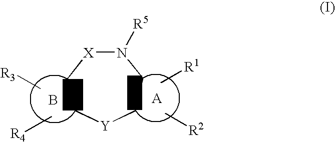
in which
X is from the group consisting of CO, CS, 502, and CH2
Y is selected from the group consisting of O, S, SO, SO₂, CH₂, C═O, C═CH₂, N—R₆, and CH—OR₆, CH—NR₆R₉, and C═CH—CO—R7
A and B are independently selected from the group consisting of 5- or 6-membered rings, aromatics such as phenyl or heteroaromatics selected from the group consisting of furan, thiophene, pyrrole, oxazole, thiazole, imidazole, pyrazole, isoxazole, isothiazole, 1,2,3-oxathiazole, 1,2,3-triazole, pyridine, pyridazine, pyrimidine and pyrazine;
R1 R2, R3, R4 are independently selected from the group consisting of H, halogen, CF₃, NO₂, NR9RIO, CN, COOH, (CH₂)m-CONR9RIO, C1-6 alkyl, OH, O—C1-6 alkyl, O-cyclopropyl, O—(CH₂)₂—O—CI-6 alkyl, O—(CH₂)₂—NR9RIO, O—CONHR9, CH₂-Z-R8, COR9, CR9RI3RI4, SR9, SOR15, SO₂R15, CR9NOR9, CR9NNR9R10, a Q-(CH2)nCONHOH group, or a 5- or 6-membered ring selected from the group consisting of furan, thiophene, pyrrole, oxazole, thiazole, imidazole, pyrazole, isoxazole, isothiazole, 1,2,3-oxathiazole, 1,2,3-triazole, pyridine, pyridazine, pyrimidine, pyrazine, morpholine, thiomorpholine, piperidine, and pyrrolidine; R5 and R6 can independently be selected from the group consisting of H, C1-6 alkyl, and QI-(CH₂)nCONHOH
R7 is a NH—(CH₂)nCONHOH group
R8 is a (CH₂)p-R11 group where R11 can be a methyl or a hydroxyl group
Z is selected from the group consisting of O, NR12, and S
Q can be a chemical bond, or can be selected from the group consisting of —O—, —S—, —NR12-, —NR9CO—, —CONR9-, —W—, —COW— where W represents a group selected from piperidine or pyrrolidine
Q1 can be a bond or a —CO—
R9 and RI0 can independently be H or a C1-6 alkyl group
R12 is H or the R8 group
R13 and R14 can be either both a fluorine atom or oxygen atoms linked together by an alkyl chain consisting of 2 or 3 CH2
R15 is a C1-6 alkyl
n is an integer between 2 and 9
m is an integer between 0 and 2
p is an integer between 0 and 5 with the limitations that:
one group containing a (CH2)nCONHOH hydroxamate and only one must always be present in the molecule
when X═CO and A and B both represent a benzene group, R3 and R4 cannot signify Q-(CH₂)nCONHOH
their optical isomers, enantiomers or diastereoisomers, and mixtures thereof, either as racemes or in various mutual ratios.

2. The compounds as claimed in claim 1, having the general formula (I), in which:
X is selected from the group consisting of CO and SO₂
Y is selected from the group consisting of O, S, SO₂, CH₂, C═O, C═CH₂, N—R6, C═CH— and CO—R7;
A and B are independently selected from the group consisting of 5- or 6-membered rings, aromatics such as phenyl or heteroaromatics selected from the group consisting of thiophene, pyrrole, oxazole, thiazole, imidazole, pyrazole, isoxazole, isothiazole, 1,2,3-oxathiazole, 1,2,3-triazole, and pyridine;
R1, R2, R3, R4 are independently selected from the group consisting of H, halogen, CF₃, NO₂, NR9RIO, CN, COOH, (CH₂)m—CONR9R1O, C1-6 alkyl, OH, O—C1-6 alkyl, O-cyclopropyl, O—(CH₂)₂—O—C1-6 alkyl, O—(CH₂)₂—NR9R1O, O—CONHR9, CH2-Z-R8, COR9, CR9RI3R14, SR9, SORI5, SO₂R15, CR9NOR9, CR9NNR9R10, and a Q-(CH₂)nCONHOH group;
R5 and R6 can independently be a group selected from H, C1-6 alkyl, and QI-(CH₂)ₙCONHOH
R7 is a NH—(CH₂)ₙCONHOH group
R8 is a (CH₂)p-R11 group where R11 can be a methyl or a hydroxyl group
Z is selected from the group consisting of O, NR12, and S
Q can be a chemical bond, or can be chosen from the group —O—, —S—, —NR12-, NR9CO—, —CONR9-, and —COW—, where W represents a group chosen from piperidine or pyrrolidine
Q1 can be a bond or —CO—
R9 and R10 can independently be H or a C1-6 alkyl group
R12 is H or the R8 group
R13 and R14 can be either both a fluorine atom or oxygen atoms linked together by an alkyl chain consisting of 2 or 3 CH2
R15 is a C1-6 alkyl
n is an integer between 2 and 9
m is an integer between 0 and 2
p is an integer between 0 and 5
with the limitations that:
one group containing a (CH₂)nCONHOH hydroxamate and only one must always be present in the molecule
when X═CO and A and B both represent a benzene group, R3 and R4 cannot signify Q-(CH₂)nCONHOH.

3. The compounds as claimed in claim 2, of general formula (I) in which:
X is selected from the group consisting of CO, and SO2
Y is selected from the group consisting of O, S, SO, SO₂, C═O, and N—R6
A and B are independently selected from 5- or 6-membered rings, aromatics such as phenyl or heteroaromatics selected from the group consisting of thiophene, pyrrole, oxazole, thiazole, imidazole, pyrazole, isoxazole, isothiazole, 1,2,3-oxathiazole, 1,2,3-triazole, and pyridine
R1 R2, R3, R4 are independently selected from the group consisting of H, halogen, CF₃, NO₂, NR9RIO, CN, CI-6 alkyl, OH, O—CI-6 alkyl, O—(CH₂)₂—NR9RIO, CH₂-Z-R8, COR9, CR9RI3RI4, 5R9, SORI5, SO2RI5, a Q(CH₂)nCONHOH group —R5 and R6 can independently be a group selected from H, C1-6 alkyl, and QI-(CH₂)nCONHOH
R8 is a (CH2)p-R11 group where R11I can be a methyl or a hydroxyl group
Z is selected from the group consisting of O, NR12, and S
Q can be a chemical bond, or can be selected from the group consisting of —O—, —S—, —NR12-, —NR9CO—, —CONR9-, and —COW—, where W represents a group selected from piperidine or pyrrolidine
Q1 can be a bond or —CO—
R9 and R10 can independently be H or a C1-6 alkyl group
R12 is H or the R8 group
R13 and R14 can be either both a fluorine atom or oxygen atoms linked together by an alkyl chain consisting of 2 or 3 CH₂ groups
R15 is a C1-6 alkyl
n is an integer between 2 and 6
p is an integer between 0 and 5 with the limitations that:
one group containing a (CH₂)nCONHOH hydroxamate and only one must always be present in the molecule
when X═CO and A and B both represent a benzene group, R3 and R4 cannot signify Q-(CH₂)nCONHOH.

4. The compounds, as claimed in claim 3:
6-(1′-Oxo-5,11-dihydro-dibenzo[b,e][1,4]diazepin-10-yl)-hexanoic acid hydroxyamide
6-(11-Oxo-I1H-dibenzo[b,f][1,4]thiazepin-10-yl)-hexanoic acid hydroxyamide
6-(8-Methoxy-11I-oxo-5,11-dihydro-dibenzo[b,e][1,4]diazepin-10-yl)-hexanoic acid hydroxiamide
6-(8-Chloro-11-oxo-5,11-dihydro-dibenzo[b,e][1,4]diazepin-10-yl)-hexanoic acid hydroxyamide
6-(8-Chloro-11-oxo-11H-dibenzo[b,f][1,4]thiazepin-10-yl)-hexanoic acid hydroxyamide
6-(8-Methyl-11I-oxo-5,11-dihydro-dibenzo[b,e][1,4]diazepin-10-yl)-hexanoic acid hydroxyamide
6-(5,5,11-Trioxo-5,11-dihydro-5X⁶-dibenzo[b,f][1,4]thiazepin-10-yl)-hexanoic acid hydroxyamide
6-(8-Methoxy-5,5,11-trioxo-5,11-dihydro-5X⁶-dibenzo[b,f][1,4]thiazepin-10-yl)-hexanoic acid hydroxyamide
6-(8-Chloro-5,5,11-trioxo-5,11-dihydro-5X⁶-dibenzo[b,f][1,4]thiazepin-10-yl)-hexanoic acid hydroxyamide
6-(8-Methoxy-5,11-dioxo-5,11-dihydro-5X⁴-dibenzo[b,f][1,4]thiazepin-10-yl)-hexanoic acid hydroxyamide
6-(11-Oxo-11H-dibenzo[b,f][1,4]oxazepin-10-yl)-hexanoic acid hydroxyamide
6-(8-Methoxy-11-oxo-11H-dibenzo[b,f][1,4]oxazepin-10-yl)-hexanoic acid hydroxyamide
6-(8-Chloro-11-oxo-11H-dibenzo[b,f][1,4]oxazepin-10-yl)-hexanoic acid hydroxyamide
7-(11-Oxo-11H-dibenzo[b,f][1,4]oxazepin-10-yl)-heptanoic acid hydroxyamide
6-(5-Oxo-5,11-dihydro-benzo[b]pyrido[2,3-e][1,4]diazepin-6-yl)-hexanoic acid hydroxyamide
6-(6,7-Dichloro-I0-oxo-4H,1OH-2-thia-4,9-diaza-benzo[t]azulen-9-yl)-hexanoic acid hydroxyamide
6-(8-Methoxy-5-oxo-5,11-dihydro-benzo[b]pyrido[2,3-e][1,4]diazepin-6-yl)-hexanoic acid hydroxyamide
6-(8,9-Dimethyl-5-oxo-5,11-dihydro-benzo[b]pyrido[2,3-e][1,4]diazepin-6-yl)-hexanoic acid hydroxyamide
6-(8-Dimethylamino-10,10-dioxo-5,10-dihydro-10X⁶-thia-5,11-diaza-dibenzo[a,d]cyclohepten-11-yl)-hexanoic acid hydroxyamide 6-(3-Methoxy-10,10-dioxo-5,10-dihydro-10X⁶-thia-5,11-diazadibenzo[a,d]cyclohepten-11-yl)-hexanoic acid hydroxyamide
6-(10,10-Dioxo-5,10-dihydro-10X⁶-thia-5,11-diaza-dibenzo[a,d]cyclohepten-11-yl)hexanoic acid hydroxyamide
6-(10,10-Dioxo-1OH-5-oxa-10X⁶-thia-11-aza-dibenzo[a,d]cyclohepten-11-yl)hexanoic acid hydroxyamide
6-(8-Amino-10,10-dioxo-5,10-dihydro-10X⁶-thia-5,11-diazadibenzo[a,d]cyclohepten-11-yl)-hexanoic acid hydroxyamide 6-(2-Fluoro-10,10-dioxo-5,10-dihydro-10X⁶-thia-5,11-diazadibenzo[a,d]cyclohepten-11-yl)-hexanoic acid hydroxyamide 6-(8-Dimethylamino-3-hydroxy-10,10-dioxo-5,10-dihydro-10X⁶-thia-5,11-diaza-dibenzo[a,d]cyclohepten-11-yl)-hexanoic acid hydroxyamide 6-(8-Dimethylamino-3-methoxy-10,10-dioxo-5,10-dihydro-10X⁶-thia-5,11-diazadibenzo[a,d]cyclohepten-11-yl)-hexanoic acid hydroxyamide 6-(7-Methyl-10,10-dioxo-5,10-dihydro-10X⁶-thia-5,11-diazadibenzo[a,d]cyclohepten-11-yl)-hexanoic acid hydroxyamide 6-(2-Methoxy-10,10-dioxo-5,10-dihydro-10X⁶-thia-5,11-diazadibenzo[a,d]cyclohepten-11-yl)-hexanoic acid hydroxyamide 6-(7-Methoxy-10,10-dioxo-5,10-dihydro-10X⁶-thia-5,11-diaza-dibenzo[a,d]cyclohepten-11-yl)-hexanoic acid hydroxyamide 6-(11-Methyl-10,10-dioxo-10,11-dihydro-5H-I0X⁶-thia-5,11-diazadibenzo[a,d]cyclohepten-7-yloxy)-hexanoic acid hydroxyamide 6-(4-Amino-10,10-dioxo-5,10-dihydro-10X⁶-thia-5,11-diazadibenzo[a,djcyclohepten-11-yl)-hexanoic acid hydroxyamide 6-(I0-Oxo-4H,1OH-2-thia-4,9-diaza-benzo[t]azulen-9-yl)-hexanoic acid hydroxyamide
6-(6,7-Dichloro-10-oxo-4H,1OH-2-thia-4,9-diaza-benzo[f]azulen-9-yl)-hexanoic acid hydroxyamide
N-Hydroxy-4-[1-(11-oxo-I0,11-dihydro-5H-dibenzo[b,e][1,4]diazepine-6-carbonyl)-piperidin-4-yl]-butyramide.

5. A pharmaceutical composition comprising the compounds as claimed in claim 1 as histone deacetylase inhibitors.

6. A method for treating inflammatory disorders, diabetes, complications of diabetes, homozygotic thalassaemia, fibrosis, cirrhosis, acute promyelocytic leukaemia (APL), transplant rejection, auto-immune diseases, protozoan infections and tumorous pathologies wherein the compounds according to claim 1 are administered to the patient.

7. A method for treating tumorous pathologies wherein compounds according to claim 1 are administered to the patient.

8. A method for treating tumours wherein compounds according to claim 1 are administered to the patient in combination with one or more active principles selected from the group consisting of chemotherapeutic agents.

9. A method for treating tumours wherein compounds according to claim 1 are administered to the patient in combination with radiotherapeutic treatments.

10. A method according to claim 8, wherein said chemotherapeutic agents are selected from the group consisting of conventional cytotoxic agents, demethylating agents, cyclin dependent kinase inhibitors, differentiating agents, signal transduction modulators, HSP-90 antagonists, and proteasome inhibitors.

11. A method according to claim 10 wherein said conventional cytotoxic agents are selected from the group consisting of Fludarabine, gemcitabine, decitabine, paclitaxel, carboplatin and Topo I/II inhibitors, to include Etoposide, Irinotecan, Topotecan, T-128 and Anthracyclines such as Doxorubicin, Sabarubicin, Daunorubicin; the demethylating agents: 5-aza-2′-deoxycytidine (5-aza-dC), 5-azacytidine; the cyclin dependent kinase inhibitors: flavopiridol, olomoucin, roscovitin, purvalanol B, GW9499, GW51 81 CGP60474, CGP7451 4, AG1 2286, AG1 2275, Staurosporine, UCN-OI; the differentiating agents: retinoic acid and derivatives (All Trans Retinoic Acid, ATRA), 13-cis retinoic acid (CRA), PMA (phorbol myristate acetate); the signal transduction modulators: TRAIL, imatinib mesylate, LY-294002, bortezomib; the HSP-90 antagonists: geldanamycin and its analogues (17-AAG); the proteasome inhibitors: lactacystine, MG132, bortezomib (Velcade™).

12. Pharmaceutical compositions containing as active principle a compound of general formula (I) as claimed in claim 1 for treating inflammatory disorders, diabetes, complications of diabetes, homozygotic thalassaemia, fibrosis, cirrhosis, acute promyelocytic leukaemia (APL), transplant rejection, auto-immune diseases, protozoan infections and tumorous pathologies.
